(19) United States
(12) Patent Application Publication
(10) Pub. No.: US 2002/0170080 A1
(43) Pub. Date: Nov. 14, 2002

(54) Alpha-tocopherol transfer protein knockout animals

(75) Inventors: Robert V. Farese JR., San Francisco, CA (US); Yuko Terasawa, Campbell, CA (US); Maret G. Traber, Corvallis, OR (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/001,278

(22) Filed: Nov. 1, 2001

Related U.S. Application Data
(60) Provisional application No. 60/245,302, filed on Nov. 2, 2000.

(51) Int. Cl. A01K 67/027; C12Q 1/68; C12N 9/10

(52) U.S. Cl. 800/14; 800/15; 800/16; 800/17; 800/18; 435/6; 435/320.1; 435/193

(57) ABSTRACT

This invention provides knockout animals comprising a disruption in one or both alleles of the gene encoding alpha-tocopherol transfer protein (TTP). The knockout animals provide good model systems for atherosclerosis.

### CROSS-REFERENCE TO RELATED APPLICATIONS

This, application claims priority to and benefit of U.S. Ser. No. 60/245,302 filed on Nov. 2, 2000, which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with the Government support under Grant No. HL1633, awarded by the National Institutes of Health. The Government of the United States of America may have certain rights in this invention.

### FIELD OF THE INVENTION

This invention relates to the field of antioxidants and the role that antioxidants play in disease development. In particular this invention pertains provides alapha-tocopherol transfer protein knockout animals that are useful models for a variety of disease states (e.g. atherosclerosis) associated with oxidative damage and antioxidant activity.

### BACKGROUND OF THE INVENTION

The major form of vitamin E in human plasma and tissues is α-tocopherol (10). α-Tocopherol enrichment of plasma and tissues is mediated by the α-tocopherol transfer protein (α-TTP), a cytosolic lipid-transfer protein expressed in the liver (Catignani and Bieri (1977) Biochim. Biophys. Acta 497: 349-357; Traber and Arai (1999) Annu. Rev. Nutr. 19: 343-355; Sato et al. (1993) J. Biol. Chem. 268: 17705-17710; Arita et al. (1995) Biochem. J. 306: 437-443). Although the mechanism is unknown (Arita et al. (1997) Proc. Natl. Acad. Sci., USA, 94: 12437-12441), α-TTP is believed to selectively transfer α-tocopherol from lipoproteins taken up by hepatocytes via the endocytic pathway to newly secreted lipoproteins, which facilitate its delivery to peripheral tissues (Traber and Arai (1999) Annu. Rev. Nutr. 19: 343-355). Humans with α-TTP gene defects have extremely low plasma α-tocopherol concentrations and develop severe neurodegenerative disease unless they are treated with high doses of vitamin E (Sokol et al. (1988) J. Lab. Clin. Med. 111: 548-559; Ouahchi et al. (1995) Nat. Genet. 9: 141-145; Hentati et al. (1996) Ann. Neurol. 39: 295-300).

Oxidative modification of lipoproteins (e.g., low density lipoproteins) has been hypothesized to play a key role in the pathogenesis of atherosclerosis (Steinberg et al. (1989) N. Engl. J. Med. 320: 915-924; Steinberg (1997) Circulation, 95: 1062-1071). Because vitamin E is the most potent lipid-soluble antioxidant normally found on lipoproteins in the plasma, there is strong interest in the relationship between vitamin E levels and the development of atherosclerosis. In animal models and human clinical trials, studies of the effects of vitamin E supplementation on atherosclerosis have yielded conflicting results (Upston, et al. (1999) FASEB J. 13: 977-994; Yusuf et al. (2000) N. Engl. J. Med. 342: 154-160; Chan (1998) J. Nutr. 128: 1593-1596; Keaney et al.(1999) FASEB J. 13: 965-976; Praticò et al. (1998) Nat. Med. 4: 1189-1192; Shaish et al. (1999) Arterioscler. Thromb. Vasc. Bigl. 19: 1470-1475), and little is known about the effects of vitamin E deficiency on atherosclerosis development (Sulkin and Sulkin (1960) Proc. Soc. Exp. Biol. Med. 103: 111-115).

### SUMMARY OF THE INVENTION

This invention provides knockout animals that are good models for vitamin E deficiency and associated pathologies (e.g. atherosclerosis, various neurologic ataxias, etc.). The animals comprise a disruption of one or both alleles of the gene encoding α-tocopherol transfer protein (Ttpa). When crossed with an animal showing reduced expression levels of Apo E protein, offspring are produced that are exceptionally good models of atherosclerosis and associated pathologies.

Thus, in one embodiment, this invention provides a knockout mammal (e.g., an equine, a bovine, a rodent, a porcine, a lagomorph, a feline, a canine, a murine, a caprine, an ovine, a non-human primate, etc.) comprising a disruption in an endogenous α-tocopherol transfer protein gene (Ttpa), where the disruption results in the knockout mammal exhibiting a decreased level of a-tocopherol transfer protein (α-TTP) as compared to a wild-type animal. In preferred embodiments, the disruption is an insertion, a deletion, a frameshift mutation, a substitution (e.g. a point mutation), or a stop codon. In particularly preferred embodiments, the disruption is an insertion of an expression cassette into the endogenous Ttpa gene. The expression cassette can express a selectable marker (e.g. a neomycin phosphotransferase gene). In certain preferred embodiments, the expression cassette is inserted into exon 1 of the endogenous Ttpa gene. This disruption can be present in a somatic and/or a germline cell and the animal can be heterozygous, homozygous, or chimeric (heterozygous or homozygous) for the disruption.

In certain particularly preferred embodiments, the mammal further comprises a second recombinantly disrupted gene (e.g. a disruption that prevents the expression of a functional polypehtide from the disrupted second gene). The mammal can be heterozygous or homozygous fcir the disrupted second gene. Preferred disruptions include, but are not limited a disrupted apo E gene, or a disrupted APP gene.

In still another embodiment, this invention provides a mammalian model of atherosclerosis. The model comprises a rodent (e.g. a mouse or a rat) comprising a disruption in an endogenous a-tocopherol transfer protein gene (Ttpa), where the disruption results in the knockout rodent exhibiting decreased levels of a-tocopherol transfer protein (α-TTP) as compared to a wild-type animal; and where the rodent exhibits reduced expression of apo E as compared to a healthy wild type rodent of the same species. In preferred embodiments, the rodent is the F1 progeny of a cross between a rodent comprising a disruption in an endogenous α-tocopherol transfer protein gene and a mammal showing reduced expression of apo E as compared to a healthy wild type rodent of the same species. The rodent can be heterozygous or homozygous for the disruption in the endogenous α-tocopherol transfer protein gene. In certain preferred embodiments, the rodent comprises a disruption (e.g. a recombinantly introduced disruption) in an endogenous apo E gene, where the disruption results in the knockout rodent exhibiting decreased levels of apo E as compared to a wild-type animal. The rodent can be homozygous or heterozygous for the disruption in an endogenous apo E gene. In certain particularly preferred embodiments, the rodent is homozygous for the disruption in an endogenous α-tocopherol transfer protein gene and homozygous for the disruption in an endogenous apo E gene. In preferred embodiments, the disruption in the (α-tocopherol transfer protein gene and/or the disruption in the apo E gene is a deletion, a frameshift mutation, a substitution (e.g. a point mutation), or a stop codon.

In still another embodiment, this invention provides a knockout rodent (e.g., a mouse, a rat, etc.) comprising a disruption in an endogenous α-tocopherol transfer protein gene (Ttpa) wherein said disruption results in said knockout rodent exhibiting decreased levels of a-tocopherol transfer protein (α-TTP) as compared to a wild-type animal. In preferred embodiments, the disruption is an insertion, a deletion, a frameshift mutation, or a stop codon. In particularly preferred embodiments, the disruption comprises an insertion of an expression cassette (e.g. as described above) into the endogenous Ttpa gene. In particularly preferred embodiments, the expression cassette is inserted into exon 1 of the endogenous Ttpa gene. The disruption can be in a somatic and/or a germline cell. The rodent can be hoinozygous or heterozygous for the disruption. In particularly preferred embodiments, the further comprises a gene that expresses a heterologous protein and/or a second recombinantly disrupted gene. When the rodent comprises a second recombinantly disrupted gene, the disruption preferably reduces or eliminates expression of a functional protein for that disrupted gene. Again, the second disruption can be in a somatic and/or a germline cell and the cell can be heterozygous or homozygous for the disruption. A preferred second gene includes, but is not limited to an apo E gene, or an APP gene.

In still another embodiment, this invention provides a nucleic acid for disrupting an α-tocopherol transfer protein gene. The nucleic acid typically includes α-tocopherol transfer protein gene sequences that undergo homologous recombination with an endogenous a-tociopherol transfer protein gene; and a nucleic acid sequence that, when introduced into an α-tocopherol transfer protein gene inhibits expression of the (α-tocopherol transfer protein gene. The α-tocopherol transfer protein gene sequence(so that undergo homologous recombination with an endogenous a-tocopherol transfer protein gene can be one or more nucleic acid sequences (e.g. sequences flanking a nucleic acid encoding a disruption (e.g., an expression cassette encoding a selectable marker)). The α-tocopherol transfer protein gene sequences that undergo homologous recombination with an endogenous a-tocopherol transfer protein gene are typically at least 5 contiguous nucleotides, more typically at least 10 contiguous nucleotides, most typically at least 15 or 20 contiguous nucleotides, preferably at least 30 contiguous nucleotides, more preferably at least 50 contiguous nucleotides and most preferably at lease 100 contiguous nucleotides (of α-tocopherol transfer protein gene sequence) in length. In particularly preferred embodiments, nucleic acid for disrupting an α-tocopherol transfer protein gene, when introduced into an a-tocopherol transfer protein gene, creates a disruption that is an insertion, a deletion, a frameshift mutation, a substitution (e.g. a point mutation), or a stop codon. In certain most preferred embodiments, the disruption comprises an insertion of an expression cassette into the endogenous Ttpa gene. The expression cassette preferably comprises a selectable marker (i.e. a nucleic acid encoding a selectable marker, e.g. neomycin phosphotransferase gene). In one most preferred embodiment the nucleic acid comprises Ttpa nucleic acid sequences flanking a nucleic acid encoding a Ttpa disruption. The nucleic acid is preferably present in a vector.

In another embodiment, this invention provides a nucleic acid (e.g. DNA, RNA, etc.) comprising a nucleic acid encoding a disrupted a-tocopherol transfer protein gene (Ttpa) wherein the disruption prevents the expression of a functional α-tocopherol transfer protein α-TTP) from the nucleic acid. The disruption is typically insertion, a deletion, a frameshift mutation, a substitution, or a stop codon. In a particularly preferred embodiment, the nucleic acid is present in a mammalian cell.

Also provided is a mammalian cell (e.g., an equine cell, a bovine cell, a rodent cell, a porcine cell, a lagomorph cell, a feline cell, a canine cell, a murine cell, a caprine cell, an ovine cell, a non-human primate cell, a human cell, etc.) comprising a disruption in an endogenous a-tocopherol transfer protein gene (Ttpa) wherein the disruption results in the cell exhibiting decreased levels of a-tocopherol transfer protein α-TTP) as compared to a wild-type animal.

Definitions

The terms “polypeptide”, “oligopeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide. Proteins also include glycoproteins (e.g. histidine-rich glycoprotein (HRG), Lewis Y antigen (Le^{Y}), and the like.).

The terms “nucleic acid”, or “oligonucleotide” or grammatical equivalents herein refer to at least two nucleotides covalently linked together. Nucleic acids of the present invention are single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Letsinger et al. (1988) J. Am. Chem. Soc. 110:4470; Letsinger et al. (1994) Nucleoside & Nucleotide 13:1597; Chapters 2 and 3, ACS Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ACS Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “residue” as used herein refers to natural, synthetic, or modified amino acids.

The term “α-tocopherol transfer protein” refers to a cytosolic lipid-transfer protein α-TTP) that is expressed in the liver. It is believed that α-TTP selectively transfers α-tocopherol, the major form of vitamin E in human plasma and tissues, from lipoproteins taken up by hepatocytes to newly secreted lipoproteins. These newly secreted lipoproteins transfer the α-tocopherol to peripheral tissues.

The term “gene” refers to a DNA sequence that comprises control and coding sequences necessary for the production of a polypeptide or precursor. The polypeptide can be encoded by a fall length coding sequence or by any portion of the coding sequence so long as the desired activity of the polypeptide is retained.

The term “Ttpa” is the gene symbol for the α-tocopherol transfer protein gene. When the gene symbol is followed by +/+ (Ttpa^{+/+}) that indicates that an organism contains two wild type alleles of the α-tocopherol transfer protein gene. When the gene symbol is followed by +/− (Ttpa^{+/-}) that indicates that an organism contains one wild-type and one disrupted allele of the α-tocopherol protein gene. When the gene symbol is followed by −/− (Ttpa^{-/-}) that indicates that the organism contains two disrupted alleles of the α-tocopherol transer protein gene.

The term “α-TTP” is a shorthand designation for and used interchangeably herein for the α-tocopherol transfer protein.

The term “endogenous α-TTP gene” refers to the wild-type gene that is found at its normal locus or position on a chromosome in a cell.

A “recombinant expression cassette” or simply an “expression cassette” is a nucleic acid construct, generated recombinantly or synthetically, with nucleic acid elements that are capable of effecting expression of a gene or cDNA in hosts compatible with such sequences. Expression cassettes typically include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein.

The term “selectable marker” refers to a nucleotide sequence that encodes a protein and that confers either a positive or negative selective advantage to a cell expressing that marker. For example, an expression cassette comprising a selectable marker could comprise the neomycin phosphotransferase (“neo”) gene operatively linked to a promoter and polyadenylation signal. Cells carrying and expressing the neo gene exhibit resistance to the selecting agent G418. Other genes which confer a positive selective advantage include, but are not limited to, the bacterial hygromycin G phosphotransferase (“hyg”) gene which confers resistance to the antibiotic hygromycin, and the bacterial xanthine-guanine phosphoribosyl traiisferase (“gpt”) gene which confers the ability to grow in the presence of mycophenolic acid. Examples of negative selectable markers include but are not limited to the herpes simplex virus thymidine kinase (“HSV-tk”) gene, the product of which is cytotoxic to cells when cells are grown in the presence of ganccyclovir or acyclovir, and the dt gene, which selects against cells capable of expressing the diptheria toxin.

The term “disruption” refers to a modification of a nucleic acid that encodes a protein or a modification of regulatory domains associated with a nucleic acid that encodes a protein such that the nucleic acid does not produce its wild-type gene product. Disruptions include, but are not limited to insertions, deletions, substitutions (e.g. point mutations) and the like. Preferred disruptions include, but are not limited to: an insertion of nucleotides that alters the reading frame of the subject nucleic acid (e.g. wild-type gene); an insertion of nucleotides that encode a heterologous protein (e.g. a selectable marker); a deletion of nucleotides that alters the reading frame of the subject nucleic acid; a deletion of nucleotides that removes portions of or complete exons, introns, splice junctions, or regulatory sequences; a modification that introduces a premature stop codon, and the like.

The term “decreased levels of α-TTP protein” when used in reference to a TTP knockout animal refers to a detectable difference of between the amount of α-TTP protein in a cell, fluid, or tissue of the knockout animal compared to the α-TTP protein in the same cell, fluid, or tissue of an animal lacking the “knockout”. In preferred embodiments, the difference is statistically significant (e.g. at greater than 80%, preferably greater than about 90%, more preferably greater than about 98%, and most preferably greater than about 99% confidence level). In particularly preferred embodiments animals that are heterozygous for the disrupted gene will express about α-TTP protein at about 50% of the level observed in the same cell, tissue, or fluid obtained from animals that are homozygous for the wild-type gene. In preferred embodiments, the α-TTP protein level in cells, fluids, or tissues of animals that are homozygous for the disrupted gene will preferably be in the range of 1.4% to 35% of the level observed in the same tissue from an animal that is homozygous for the wild-type gene.

The term “wild-type” refers to a gene or gene product, which has the characteristics of that gene or gene product when isolated from a naturally occurring source. A wild-type gene is that which is most frequently observed in a population and is thus arbitrarily designated the “normal” or “wild-type” form of the gene. In contrast, the terms “modified”, “mutant”, or “disrupted” refers to a gene or gene product which displays modifications in sequence and/or functional properties (i.e. altered characteristics) when compared to the wild-type gene or gene product. It is noted that naturally-occurring mutants can be isolated; these are typically identified by the fact that they have altered characteristics when compared to the wild-type gene or gene product.

The term “knockout” refers to an animal in which normal expression of a functional gene is reduced and/or eliminated. In preferred embodiments, this is done by deleting all or a part of a gene, or by inserting a nucleic acid encoding a stop codon, or a heterologous polypetide. A knockout includes both the heterozygote animal (i.e., one defective allele and one wild-type allele) and the homozygous mutant (i.e., two defective alleles).

The term “operably linked” as used herein refers to linkage of a promoter (or other regulatory sequences) to a nucleic acid sequence such that the promoter (or other regulatory sequences) mediates/controls transcription of the nucleic acid sequence.

The term “heterologous” as it relates to nucleic acid sequences such as coding sequences and cortrol sequences, denotes sequences that are not normally associated with a region of a recombinant construct, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

The term “recombinantly disrupted” refers to the disruption of a gene by the introduction or recombination of that gene with a heterologous nucleic acid.

### BRIEF DESCRIPTION OF THE DRAWINGS

FIGS. 1A through 1D illustrate the generation of Ttpa^{-/-} mice. FIG. 1A illustrates the strategy for disrupting the Ttpa gene. Upon homologous recombination of the targeting vector with the Ttpa locus, lacZ (β-galactosidase) and neo genes are inserted into the 5′ untranslated sequences of exon 1, resulting in the deletion of the Ttpa translational start codon. A, B, and C represent primers used for PCR genotyping. FIG. 1B shows a Southern blot analysis of genomic DNA from offspring of heterozygous intercrosses. FIG. 1C illustrates the absence of α-TTP protein in liver homogenates of Ttpa^{-/-} mice. α-TTP protein levels were reduced by ˜50% in Ttpa^{+/-} mice. FIG. 1D shows α-Tocopherol levels in Ttpa^{+/+} apo E^{+/+} and Ttpa^{-/-} apo E^{+/+} mice and in Ttpa^{+/+} apo E^{-/-} and Ttpa^{-/-} apo E^{-/-} mice, of 7-12 months of age. Aortic α-tocopherol levels were similarly low in both Ttpa^{+/+} apo E^{-/-} and Ttpa^{-/-} apo E^{-/-} mice (0.60±0.64 vs. 0.77±0.68 nmol/g, P=0.55). Data are expressed as mean±SD.

FIGS. 2A and 2B show atherosclerotic lesion area in mouse aortas. Ttpa^{+/+} apo E^{+/-} (n=20), Ttpa^{+/-} apo E^{+/-} (n=19), and Ttpa^{-/-} apo E^{+/+} (n=21) mice were killed at age 30 weeks. FIG. 2A shows total aortic lesion area (mean±SD). * P=0.005, ANOVA with Tukey test. FIG. 2B shows regional aortic lesion area (mean±SD). * P=0.002, **P=0.03, ANOVA with Tukey test.

FIG. 3 shows the morphology of aortic lesions from proximal aortic roots. Representative section from the aortic root (at the level of the first coronary) showing lesions in Ttpa^{+/+} apo E^{-/-} mouse at low (4) (Panel A) and high magnification detail of lower right profile in A (20×) (Panel B). Representative section at the aortic root lesion from Ttpa^{-/-} apo E^{-/-} mouse at low (4×) (Panel C) and high magnification detail of lower right profile in C (20×) (Panel D). Lesions in Ttpa^{-/-} apo E^{-/-} mice show more complex features, including a large necrotic core (NC), numerous needle-shaped lucencies indicative of cholesterol crystals (C), and fibrous capping (FC) from smooth muscle cells (stained red). Within the lesion core, greenish-blue staining represents proteoglycan, and yellow staining represents collagen.

FIG. 4 shows aortic F2-isoprostane levels in proximal aortas of Ttpa^{+/+} apo E^{-/-} and Ttpa^{-/-} apc E^{-/-} mice (n=10 and 11 females, respectively). Data are expressed as mean±SD. *P=0.03, Mann-Whitney rank sum test.

### DETAILED DESCRIPTION

This invention provides animals comprising a knockout of one or both alleles of an α-tocopherol transfer protein gene (Ttpa). It is believed that the α-tocopherol transfer protein functions to incorporate α-tocopherol (vitamin E) into lipoproteins secreted by the liver. The knockout animals of this invention are good models for vitamin E deficiency providing control of vitamin E levels that cannot be readily obtained using dietary animal models. The knockout animals of this invention are useful as systems in which to research the physiology of vitamin E deficiency, and/or to study diseases involving oxidative stress (e.g. atherosclerosis, cancer, neurological diseases, etc.), and/or to screen for various agents (e.g. drugs) that mediate one or more symptoms associated with vitamin E deficiency and/or oxidative stress.

In addition, the knockout animals of this invention can be crossed with other inbred and/or knockout animals to produce refined biological models of various pathologies. Thus, for example, the Ttpa knockout animals of this invention can be crossed with animals deficient in apo E (e.g. apo E knockout animals). Animals deficient in apo E have an increased susceptibility to atherosclerosis. Animals deficient in vitamin E due to a disruption in the α-tocopherol transfer protein gene, and also deficient in apo E have an increased severity of atherosclerotic lesions in the proximal aorta. The increase in lesions is associated with increased levels of isoprostanes, a marker of lipid peroxidation, in aortic tissue. These observations show that vitamin E deficiency promotes atherosclerosis in a susceptible setting and indicates that lipid peroxidation contributes to atherosclerotic lesion development. The Ttpa knockout/apo E knockout animals are thus a particularly good model of atherosclerosis.

As indicated above, the Ttpa knockout animals of this invention have a number of uses, in particular as a genetic model of vitamin E deficiency as described herein. Having demonstrated herein that inhibition of the expression of a functional Ttpa protein will still result in a viable animal, one of ordinary skill in the art, using the teaching provided herein, can routinely produce other Ttpa knockout animals.

In another embodiment, this invention provides nucleic acid sequences (transgenes) that are capable of inactivating endogenous Ttpa genes. Such transgenes preferably contain a nucleic acid sequence (e.g. a DNA sequence) that is identical to some portion of the end:genous Ttpa gene that is to be disrupted. Preferred transgenes of this invention also contain a substitution, deletion, or insertion of one or more nucleotides as compared with undisrupted alleles of the same Ttpa gene naturally-occurring in the species.

Hormologous recombination of the transgene with a Ttpa allele disrupts the expression of that allele. Such a disruption can be by a number of mechanisms including, but not limited to, interference in initiation of transcription and/or translation, by premature termination of transcription and/or translation, and/or by production of a non-functional Ttpa protein.

In one embodiment, such transgenes are derived by deleting nucleotides from the nucleic acid sequence encoding the functional Ttpa gene. Although the resultant mutated nucleic acid sequence is incapable of being transcribed and/or translated into a functional Ttpa gene product, such transgenes will have sufficient sequence homology with an endogenous Ttpa allele of a selected non-human animal such that the transgene is capable of homologous recombination with the endogenous Ttpa allele.

In a preferred embodiment, transgenes are produced by ligation of an expression cassette encoding a selectable marker into the nucleic acid sequence encoding the Ttpa gene products and/or into the nucleic acid sequence regulating transcription of the Ttpa gene product. The cassette is preferably inserted in a location such that it replaces or disrupts regions of the encoded protein required for protein functionality. The cassette is also preferably inserted in a location such that splicing out of the cassette introduces a frameshift mutation resulting in non-functional reversions. In a more preferred embodiment, an expression cassette containing portions of the LacZ gene and the Neo gene are cloned into the 5′ untranslated sequences of exon 1 such that the translational start site of the Ttpa gene is deleted.

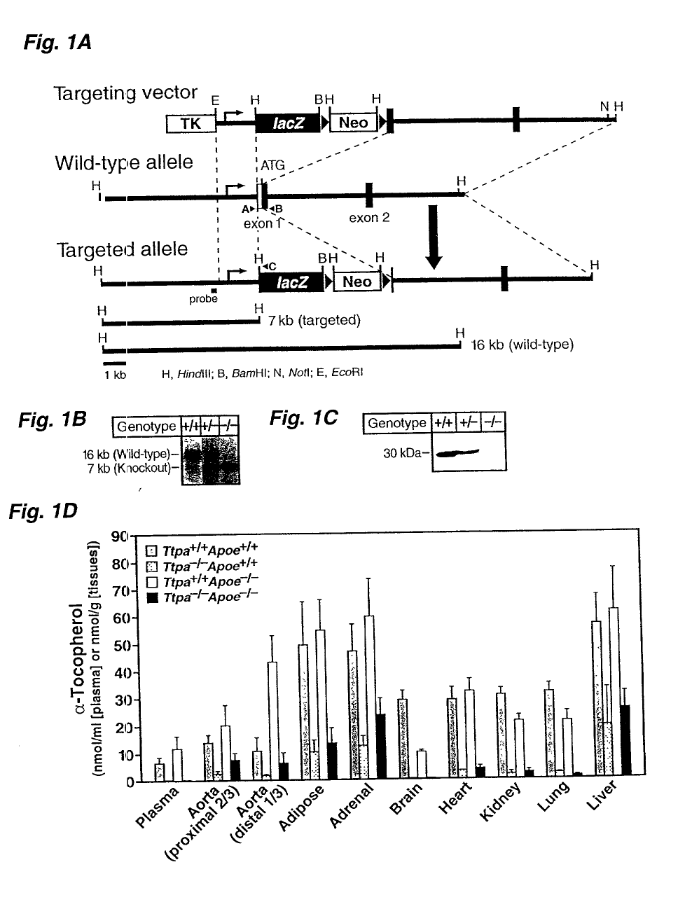

Such transgenes are preferably designed for replacement of one or more exons of the endogenous Ttpa gene (see e.g. FIG. 1A). Although insertional transgenes may also be used. replacement transgenes are preferred because they significantly reduce the likelihood of secondary recombination and reversion to the wild-type Ttpa gene.

A) Atherosclerosis Model

As indicated above, the knockout animals of this invention are particularly well suited as models of atherosclerosis. In human and animal studies, the ability of vitamin E supplementation to prevent atherosclerosis has varied, possibly because of differences in vitamin E supplementation regimens, other dietary factors, or the degree of preexisting atherosclerosis.

The animal models provided by this invention permit the effects of vitamin E deficiency on atherogenesis to be analyzed as a single modifying factor present before lesion development. The data presented herein indicate that α-TTP deficiency and associated vitamin E deficiency promote lesion formation in the proximal aorta in the setting of increased susceptibility to atherosclerosis, in the case of one model system provided herein, caused by apo E deficiency.

The apo ETTtpa knockouts of this invention provide an excellent model system in which to study atherogenesis and/or to evaluate/screen various agents for the ability to inhibit atherogenesis particular atherogenesis associated with lipid peroxidation.

B) Other Models

The Ttpa knockouts of this invention are useful themselves as models systems for a number of pathologies or can be crossed with animals exhibiting particular phenotypic traits to produce useful animal models. Thus, for example, the Ttpa knockout animals of this invention could be crossed with other animal models such as those for Alzheimer's disease (e.g. amyloid precursor protein transgenic mice) to produce useful models for oxidative stress and its impact on Alzheimer's disease.

Similarly, the animals of this invention can be crossed with animals having inhibited tumor suppressors and/or expressing oncogenes to produce animals models for evaluation of the impact of oxidative stress on cancer etiology and progression. Thus, for example, Ttpa-krockout mice can be crossed with p53 or p71 knockout mice to produce useful model systems.

The animals of this invention can also be crossed with animals exhibiting other antioxidant deficiencies, e.g. vitamin C deficient animals (see, e.g., Maeda et al. (2000) Proc. Natl. Acad. Sci., USA, 97: 841-846).

Alternatively, the Ttpa-knockout animals of this invention can be used to investigate other pathologies that involve a component of oxidative stress. For example the knockout animals of this invention are useful mouse models for investigate the effects of cigarette smoke inhalation and its relationship to lung cancer, for evaluating the effects of ozone exposure, shin UV irradiation exposure, for skin disease, for skin cancer, and the like. The animals are also useful model systems for investigation of inflammatory responses and signal transduction pathways that are sensitive to vitamin E level (e.g. protein kinase C, NADPH oxidase, expression of IL-1, IL-6, and the like).

The animals of this invention are also useful models for investigating recovery from renal injury, acute renal failure, and the like, and for investigating the role of oxidative stress in diabetes.

The animal models of this system have uses beyond that of simple research tools. For example, the knockout animals of this invention are useful in producing other useful knockout animals as explained above. In addition, they are useful systems in which to screen for agents; (e.g. small organic molecules, known drugs, gene therapy based therapeutics, etc.) that mitigate or eliminate one or more symptoms of the pathologies described above.

C) Targeting of the Disruption: Homologous Recombination

In a preferred embodiment, the present invention uses the process of homologous recombination to control the site of integration of a specific DNA sequence (transgene) into the naturally present Ttpa sequence of an animal cell and thereby disrupt that gene and prevent normal its normal expression. Homologous recombination is described in detail by Watson (1977) In: Molecular Biology of the Gene, 3rd Ed., W. A. Benjamin, Inc., Menlo Park, Calif. In brief, homologous recombination is a natural cellular process that results in the scission of two nucleic acid molecules having identical or substantially similar (i.e. “homologous”) sequences, and the ligation of the two molecules such that one region of each initially present molecule is now ligated to a region of the other initially present molecule (Sedivy (1988) Bio-Technol., 6: 1192-1196).

Homologous recombination is exploited by a number of various methods of “gene targeting” well known to those of skill in the art (see, e.g., Mansour et al. (1988) Nature, 336: 348-352; Capecchi (1989) Trends Genet. 5: 70-76; Capecchi (1989) Science 244: 1288-1292; Capecchi et al. (1989) pages 45-52 In: Current Communications in Molecular Biology, Capecchi, M. R. (ed.), Cold Spring Harbor Press, Cold Spring Harbor, N.Y.; Frohman et al. (1989) Cell 56: 145-147). Some approaches further involve increasing the frequency of recombination between two DNA molecules by treating the introduced DNA with agents which stimulate recombination (e.g. trimethylpsoralen, UV light, etc.), however, most approaches utilize various combinations of selectable markers to facilitate isolation of the transformed cells.

One such selection method is termed positive/negative selection (PNS) (Thomas and Cappechi (1987) Cell 51: 503-512). This method involves the use of two selectable markers: one a positive selection marker such as the bacterial gene for neomycin resistance (neo); the other a negative selection marker such as the herpes virus thymidine kinase (HSV-tk) gene. Neo confers resistance to the drug G-418, while HSV-tk renders cells sensitive to the nucleoside analog gangcyclovir (GANC) or 1-(2-deoxy-2-fluoro-b-d-arabinofuranosyl)-5-iodouraci21(FIAU). The DNA encoding the positive selection marker in the transgene (e.g. neo) is generally linked to an expression regulation sequence that allows for its independent transcription in embryonic stem (ES) cells. It is flanked by first and second sequence portions of at least a part of the Ttpa gene.

These first and second sequence portions target the transgene to a specific allele. A second independent expression unit capable of producing the expression product for a negative selection marker, e.g. for HSV-tk is positioned adjacent to or in close proximity to the distal end of the first or second portions of the first DNA sequence. Upon transfection, some of the ES cells incorporate the transgene by random integration, others by homologous recombination between the endogenous allele and sequences in the transgene. As a result, one copy of the targeted allele is disrupted by homologous recombination with the-transgene with simultaneous loss of the sequence encoding herpes HSV-tk gene. Random integrants, which occur via the ends of the transgene, contain herpes HSV-tk and remain sensitive to GANC or FIAU. Therefore, selection, either sequentially or simultaneously with G418 and GANC enriches for transfected ES cells containing the transgene integrated into the genome by homologous recombination.

Other strategies that select for homologous recombination events but do not use PNS may also be used. For example, a promoter that is active in ES cells is operably linked to a positive selection gene such as the bacterial neo gene whose transcription unit lacks its own polyadenylation (poly-A) signal sequence. This expression unit is targeted to an exon of the endogenous Ttpa gene. Upon homologous recombination (e.g. in the ES cell) the neo gene is transcribed independently, as above. Stable transcripts from the neo gene require the presence of a poly-A site downstream. Thus, by targeting the neo gene to an endogenous Ttpa transcription unit, homologous recombinants are linked to the poly-A site of the targeted Ttpa gene which permits transcription of a functional neo transcript and selection based upon resistance to G418.

It is possible that in some circumstances it will not be desirable to have an expressed antibiotic resistance gene incorporated into the knockout animal. Therefore, in certain preferred embodiments, one or more genetic elements are included in the knockout construct that permit the antibiotic resistance gene to be excised once the construct has undergone homologous recombination with the Ttpa gene.

The FLP/FRT recombinase system from yeast represents one such set of genetic elements (O'Gorman et al. (1991) Science 251, 1351-1355). FLP recombinase is a protein of approximately 45 kD molecular weight. It is encoded by the FLP gene of the 2 micron plasmid of the yeast Saccharomyces cerevisiae. The protein acts by binding to the FLP Recombinase target site, or FRT; the core region of the FRT is a DNA sequence of approximately 34 bp. FLP can mediate several kinds of recombination reactions including excision, insertion and inversion, depending on the relative orientations of flanking FRT sites. If a region of DNA is flanked by direct repeats of the FRT, FLP will act to excise the intervening DNA, leaving only a single FRT. FLP has been shown to function in a wide range of systems, including in the cultured mammalian cell lines CV-1 and F9, (O'Gorman et al. supra;, and in mouse ES cells, Jung et al. (1993) Science 259: 984).

The methods discussed below are capable of mutating both alleles of the cell's Ttpa gene, however, since the frequency of such dual mutational events is the square of the frequency of a single mutational event, cells having mutations in both of their Ttpa alleles will be only a very small proportion of the total population of mutated cells. It is possible to readily identify (for example through the use of Southern hybridization or other methods) whether the mutational events are single allele or dual allele events. Animals having a mutational event in a single allele may be cross-bred to produce homozygous animals (having the disruption in both alleles) if the disruption becomes incorporated in the germ line.

In a preferred embodiment, the nucleic acid molecule(s) that are to be introduced into the recipient cell contain a region of homology with a region of the Ttpa gene. In a preferred embodiment, the nucleic acid molecule will contain two regions having homology with the cell's Ttpa gene. These “regions of homology” will preferably flank the precise sequence whose incorporation into the Ttpa gene is desired.

The nucleic acid molecule(s) may be single stranded, but are preferably double stranded. The molecule(s) may be introduced to the cell as DNA molecules, as one or more RNA molecules which may be converted to DNA by reverse transcriptase or by other means. Detailed protocols for production of a Ttpa knockout animal of this invention are provided in Exaimple 1.

D) Transformation of Cells

To produce the knockout animal, cells are transformed with the construct (e.g. transgene) described above. As used herein, the term “transformed” is defined as introduction of exogenous DNA into the target cell by any means known to the skilled artisan. These methods of introduction can include, without limitation, transfection, microinjection, infection (with, for example, retroviral-based vectors), electroporation and microballistics. The term “transformed” unless otherwise indicated, is not intended herein to indicate alterations in cell behavior and growth patterns accompanying immortalization, density-independent growth, malignant transformation or similar acquired states in culture.

To create animals having a particular gene inactivated in all cells, it is preferable to introduce a knockout construct into the germ cells (sperm or eggs, i.e., the “germ line”) of the desired species. Genes or other DNA sequences can be introduced into the pronuclei of fertilized eggs by microinjection or other methods as described below. Following pronucl ear fusion, the developing embryo may carry the introduced gene in all its somatic and germ cells since the zygote is the mitotic progenitor of all cells in the embryo. Since targeted insertion of a knockout construct is a relatively rare event, it is desirable to generate and screen a large number of animals when employing such an approach. Because of this, it can be advantageous to work with the large cell populations and selection criteria that are characteristic of cultured cell systems. However, for production of knockout animals from an initial population of cultured cells, it is preferred that a cultured cell containing the desired knockout construct bre capable of generating a whole animal. This is generally accomplished by placing the cell into a developing embryo environment of some sort.

Cells capable of giving rise to at least several differentiated cell types are hereinafter termed “pluripotent” cells. Pluripotent cells capable of giving rise to all cell types of an embryo, including germ cells, are hereinafter termed “totipotent” cells. Totipotent murine cell lines (embryonic stem, or “ES” cells) have been isolated by culture of cells derived fror very young embryos (blastocysts). Such cells are capable, upon incorporation into an embryo, of differentiating into all cell types, including germ cells, and can be employed to generate animals lacking a functional Ttpa gene. That is, cultured ES cells can be transformed with a knockout construct, as described herein, and cells selected in which the Ttpa gene is inactivated through insertion of the construct within, for example, an appropriate exon (e.g. exon 1 as illustrated in Example 1).

1) Microinjection Methods

The “transgenic non-human animals” of the invention are produced by introducing “transYenes” into the germline of the non-human animal. Embryonic target cells at various developmental stages can be used to introduce transgenes. Different methods are used depending or the stage of development of the embryonic target cell.

Microinjection is one preferred method for transformation of a zygote. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) Proc. Natl. Acad. Sci. USA 82, 4438-4442). As a consequence, all cells of the transgenic non-human animal will carry the incorporated transgene. This will, in general, also be reflected in the efficient transmission of the transgene to offspring of the founder since 50% of the germ cells will harbor the transgene.

The gene sequence being introduced need not be incorporated into any kind of self-replicating plasmid or virus (Jaenisch, (1988) Science, 240: 1468-1474). Indeed, the presence of vector DNA has been found, in many cases, to be undesirable (Hammer et al. (1987) Science 235: 53; Chada et al. (1986) Nature 319: 685; Kollias et al., (1986) Cell 46: 89; Shani, (1986) Molec, Cell, Biol. 6: 2624 (1986); Chada, et al. (1985) Nature, 314: 377;; Townes et al. (198) EMBO J. 4: 1715).

Once the DNA molecule has been injected into the fertilized egg cell, the cell is implanted into the uterus of a recipient female, and allowed to develop into an animal. Since all of the animal's cells are derived from the implanted fertilized egg, all of the cells of the resulting animal (including the germ line cells) shall contain the introduced gene sequence. If, as occurs in about 30% of events, the first cellular division occurs before the introduced gene seqauence has integrated into the cell's genome, the resulting animal will be a chimeric animal.

By breeding and inbreeding such animals, it is possible to routinely produce heterozygous and homozygous transgenic animals. Despite any unpredictability in the formation of such transgenic animals, the animals have generally been found to be stable, and to be capable of producing offspring that retain and express the introduced gene sequence.

The success rate for producing transgenic animals is greatest in mice. Approximately 25% of fertilized mouse eggs into which DNA has been injected, and which have been implanted in a female, will become transgenic mice. A number of other transgenic animals have also been produced. These include rabbits, sheep, cattle, and pigs (Jaenisch (1988) Science 240: 1468-1474; Hammer et al., (1986) J. Animal. Sci, 63: 269 Hammer et al. (1985) Nature 315: 680; Wagner et al., (1984) Theriogenology 21: 29).

2) Retroviral Methods.

Retroviral infection can also be used to introduce a transgene into a non-human animal. The developing non-human embryo can be cultured in vitro to the blastocyst stage. During this time, the blastomeres can be targets for retroviral infection (Jaenich (1976) Proc. Natl. Acad. Sci USA 73: 1260-1264). Efficient infection of the blastomeres is obtained by enzymatic treatment to remove the zona pellucida (Hogan, et al. (1986) In Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner, et al. (1985) Proc. Natl. Acad. Sci. USA 82, 6927-6931; Van der Putten, et al. (1985) Proc. Natl. Acad. Sci., USA, 82, 61,486,152). Transfection is easily and efficiently obtained by culturing the blastomeres on a monolayer of virus-producing cells (Van der Putten, supra; Stewart et al. (1987) EMBO J., 6: 383-388). Alternatively, infection can be performed at a later stage. Virus or virus-producing cells can be injected into the blastocoele (Jahner et al. (1982) Nature, 298: 623-628). Most of the founders will be mosaic for the transgene since incorporation occurs only in a subset of the cells, which formed the transgenic non-human animal. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the midgestatior embryo (Jahner et al. (1982) supra).

3 ES Cell Implantation.

A third and preferred target cell for transgene introduction is the embryonic stem cell (ES). ES cells are obtained from pre-implantation embryos cultured in vitro and fused with embryos (Evans, et al. (1981) Nature, 292: 154-156; Bradley, et al. (1984) Nature, 309: 255-258; Gossler, et al. (1986) Proc. Natl. Acad. Sci., USA, 83:, 9065-9069; and Robertson, et al. (1986) Nature, 322: 445-448). Transgenes can be efficiently introduced into the ES cells a number of means well known to those of skill in the art. Such transformed ES cells can thereafter be combined with blastocysts from a non-human animal. The ES cells thereafter colonize the embryo and contribute to the germ line of the resulting chimeric animal (for a review see Jaenisch (1988) Science, 240: 1468-1474).

The DNA molecule containing the desired gene sequence may be introduced into the pluripoterit cell by any method which will permit the introduced molecule to undergo recombination at its regions of homology. Transgenes can be efficiently introduced into the ES cells by DNA ixansfection or by retrovirus-mediated transduction.

In a preferred embodiment, the DNA is introduced by electroporation (Toneguzzo et al., (1988) Nucleic Acids Res., 16: 5515-5532; Quillet et al. (1988) J. Immunol., 141: 17-20; Machy et al. (1988) Proc. Natl. Acad. Sci., USA, 85: 8027--8031). After permitting the introduction of the DNA molecule(s), the cells are cultured under conventional conditions, as are known in the art.

In order to facilitate the recovery of those cells that have received the DNA molecule containing the desired gene sequence, it is preferable to introduce the DNA containing the desired gene sequence in combination with a second gene sequence that would contain a detectable marker gene sequence. Where it is only desired to introduce a disruption into a gene, the DNA sequence containing the detectable marker sequence may itself comprise the disruption. For the purposes of the present invention, any gene sequence whose presence in a cell permits one to recognize and clonally isolate the cell may be employed as a detestable (selectable) marker gene sequence.

In one embodiment, the presence of the detectable (selectable) marker sequence in a recipient cell is recognized by hybridization, by detection of radiolabelled nucleotides, or by other assays of detection which do not require the expression of the detectable marker sequence. In one embodiment, such sequences are detected using polymerase chain reaction (PCR) or other DNA amplification techniques to specifically amplify the DNA marker sequence (Mullis et al., (1986) Cold Spring Harbor Symp. Quant. Biol. 51: 263-273; Erlich et al. EP 50,424; EP 84,796, EP 258,017 and EP 237,362; Mullis EP 201,184; Mullis et al., U.S. Pat. No. 4,683,202; Erlich U.S. Pat. No. 4,582,788; and Saiki et al. U.S. Pat. No. 4,683,194).

Most preferably, however, the detectable marker gene sequence will be expressed in the recipient cell, and will result in a selectable phenotype. Selectable markers are well known to those of skill in the art. Some examples include the hprt gene (Littlefield (1964) Science 145 :709-710), the thymidine kinase gene of herpes simplex virus (Giphart-Gassler et al. (1989) Mutat, Res., 214: 223-232), the nDtII gene (Thomas et al. (1987) Cell, 51: 503-512; Mansour et al. (1988) Nature 336: 348-352), or other genes which confer resistance to amino acid or nucleoside analogues, or antibiotics, etc.

Thus, for example, cells that express an active HPRT enzyme are unable to grow in the presence of certain nucleoside analogues (such as 6-thioguanine, 8-azapurine, etc.), but are able to grow in media supplemented with HAT (hypoxanthine, aminopterin, and thymidine). Conversely, cells which fail to express an active HPRT enzyme are unable to grow in media containing HATG, but are resistant to analogues such as 6-thioguanine, etc. (Littlefield (1964) Science, 145: 709-710). Cells expressing active thymidine kinase are able to grow in media containing HAT, but are unable to grow in media containing nucleoside analogues such as bromo-deoxyuridine (Giphart--Gassler et al. (1989) Mutat. Res. 214: 223-232). Cells containing an active HSV-tk gene are incapable of growing in the presence of gangcylovir or similar agents.

The detectable marker gene may also be any gene that can compensate for a recognizable cellular deficiency. Thus, for example, the gene for HPRT could be used as the detectable marker gene sequence when employing cells lacking HPRT activity. Thus, this agent is an example of agents may be used to select mutant cells, or to “negatively select” for cells which have regained normal function.

In preferred embodiments, the chimeric or transgenic animal cells of the present invention are prepared by introducing one or more DNA molecules into a precursor pluripotent cell, most preferably an ES cell, or equivalent (Robertson (1989) pages 39-44 In: Current communications in Molecular Biology, Capecchi, M. R. (ed.), Cold Spring Harbor Press, Cold Spring, Harbor, N.Y. -The term “precursor” is intended to denote only that the pluripotent cell is a precursor to the desired (“transfected”) pluripotent cell which is prepared in accordance with the teachings of the present invention. The pluripotent (precursor or transfected) cell may be cultured in vivo, in a manner known in the art (Evans et al., (1981) Nature 292: 154-156) to form a chimeric or transgenic animal. The transfected cell, and the cells of the embryo that it forms upon introduction into the uterus of a female are herein referred to respectively, as “embryonic stage” ancestors of the cells and animals of the present invention.

Any ES cell may be used in accordance with the present invention. It is, however, preferred to use primary isolates of ES cells. Such isolates may be obtained directly from embryos such as the CCE cell line disclosed by Robertson, E. J., In: Current Communications in Molecular Biology, Capecchi, M. R. (ed.), Cold Spring Harbor Press, Cold Spring Harbor, N.Y. (1989), pp. 39-44), or from the clonal isolation of ES cells from the CCE cell line (Schwartzberg et al. (1989) Science 212: 799-803). Such clonal isolation may be accomplished according to the method of Robertson (1987) In: Teratocarcinomas and Embryonic Stem Cells: A Practical Approach, E. J. Robertson, Ed., IRL Press, Oxford. The purpose of such clonal propagation is to obtain ES cells that have a greater efficiency for differentiating into an animal. Clonally selected ES cells are approximately 10-fold more effective in producing transgenic animals than the progenitor cell line CCE. An example of ES cell lines which have been clonally derived from embryos are the ES cell lines, AB1 (hprt+) or AB2.1 (hprt−).

The ES cells are preferably cultured on stromal cells (such as STO cells (especially SNL,76/7 STO cells) and/or primary embryonic G418 R fibroblast cells) as described by Robertson, supra. Methods for the production and analysis of chimeric mice are well known to those of skill in the art (see, e.g., Bradley (1987) pages 113-151 In: Teratocarcinomas and Embryonic Stem Cells; A Practical Approach, E. J. Robertson, ed., IRL Press, Oxford). The stromal (and/or fibroblast) cells serve to eliminate the clonal overgrowth of abnormal ES cells. Most preferably, the cells are cultured in the presence of leukocyte inhibitory factor (“lif”) (Gough et al. (1989) Reprod. Fertil., 1: 281-288; Yamamori et al. (1989) Science, 246: 1412-1416). Since the gene encoding lif has been cloned (Gough, et al. supra.), it is especially preferred to transform stromal cells with this gene, by means known in the art, and to then culture the ES cells on transformed stromal cells that secrete lif into the culture medium.

ES cell lines may be derived or isolated from any species (for example, chicken, ect.), although cells derived or isolated from mammals such as rodents, rabbits, sheep, goats, fish, pigs, cattle, primates and humans are preferred. Cells derived from rodents (i.e. mouse, rat, hamster ect.) are particularly preferred.

In fact, ES cell lines have been derived for mice and pigs as well as other animals (see, e.g., Robertson, Embryo-Derived Stem Cell Lines. In: Teratocarcinomas and Embryonic Stem Cells: A Practical Approach (E. J. Robertson, ed.), IRL Press, Oxford (1987); PCT Publication No. WO/90/03432; PCT Publication No. 94/26884. Generally these cells lines rnust be propagated in a medium containing a differentiation-inhibiting factor (DIF) to prevent spontaneous differentiation and loss of mitotic capability. Leukemia Inhibitory Factor, LIF) is particularly useful as a DIF. Other DIF's useful for prevention of ES cell differentiation include, without limitation, Oncostatin M (Gearing and Bruce (1992) The New Biologist 4: 61-65), interleukin 6 (IL-6) with soluble IL-6 receptor (sIL-6R) (Taga et al. (1989) Cell 58: 573-581), and ciliary neurotropic factor (CNTF) (Conover et al. (1993) Development 19: 559-565). Other known cytokines may also function as appropriate DIF's, alone or in combination with other DIF's.

As a useful advance in maintenance of ES cells in an undifferentiated state, a novel variant of LIF (T-LIF) has been identified (see U.S. Pat. No. 5,849,991). In contrast to the previously identified forms of LIF which are extracellular, T-LIF is intracellularly localized. The transcript was cloned from murine ES cells using the RACE technique, Frohman et al. (1988) Proc. Natl. Acad. Sci., USA, 85: 8998-9002), and subjected to sequence analysis,. Analysis of the obtained nucleic acid sequence and deduced amino acid sequence indicates that T-LIF is a truncated form of the LIF sequence previously reported in the literature. Expression of the T-LIF nucleic acid in an appropriate host cell yields a 17 kD protein that is unglycosylated. This protein is useful for inhibiting differentiation of murine ES cells in culture.

E) Production of Transgenic Animals Via the Somatic Cell Nuclear Transfer

Production of the knockout animals of this invention is not dependent on the availability of ES cells. In various embodiments, knockout animals of this invention can be produced using methods of somatic cell nuclear transfer. In preferred embodiments using such an approach, a somatic cell is obtained from the species in which the Ttpa gene is to be knocked out. The cell is transfected with a construct that introduces a disruption in the Ttpa gene (e.g. via heterologous recombination) as described herein. Cells harboring a knocked out Ttpa are selected as described herein. The nucleus of such cells harboring the knockout is then placed in an unfertilized enucleated egg (e.g., eggs from which the natural nuclei have been removed by microsurgery). Once the transfer is complete, the recipient eggs contained a complete set of genes, just as they would if they had been fertilized by sperm. The eggs are then cultured for a period before being implanted into a host mammal (of the same species that provided the egg) where they are carried to term, culminating in the berth of a transgenic animal comprising a nucleic acid construct containing one or more disrupted Ttpa genes (e.g. the disrupted Ttpa gene).

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Using methods of nuclear transfer as describe in these and other references, cell nuclei derived from differentiated fetal or adult, mammalian cells are transplanted into enucleated mammalian oocytes of the same species as the donor nuclei. The nuclei are reprogrammed to direct the development of cloned embryos, which can then be transferred into recipient females to produce fetuses and offspring, or used to produce cultured inner cell mass (CICM) cells. The cloned embryos can also be combined with fertilized embryos to produce chimeric embryos, fetuses and/or offspring.

Somatic cell nuclear transfer also allows simplification of transgenic procedures by working with a differentiated cell source that can be clonally propagated. This eliminates the need to maintain the cells in an undifferentiated state, thus, genetic modifications, both random integration and gene targeting, are more easily accomplished. Also by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

Differentiated mammalian cells are those cells that are past the early embryonic stage. More particularly, the differentiated cells are those from at least past the embryonic disc stage (day 10 of bovine embryogenesis). The differentiated cells may be derived from ectocderm, mesoderm or endoderm.

Mammalian cells, including human cells, may be obtained by well known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, ect. Moreover, the mammalian cells used for nuclear transfer may be obtained from different organs, e.g., skin, lung, pancreas, liver, stomach, intestine, heart, reproductive organs, bladder, kidney, urethra and other urinary organs, ect. These are just examples of suitable donor cells. Suitable donor cells, i.e., cells useful in the subject invention, may be obtained from any cell or organ of the body. This includes all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures. Again the present invention is novel because differentiated cell types are used. The present invention is advantageous because the cells can be easily propagated, genetically modified and selected in vitro.

As indicated above, once the Ttpa gene has been knocked out in a somatic cell the nucleus is transferred to an oocyte, preferably to a mammalian oocyte. Suitable mammalian sources for oocytes include, but are not limited to sheep, cows, pigs, horses, rabbits, guinea pigs, mice, hamsters, rats, non-human primates, ect. Methods for isolation of oocytes are well known in the art.

The oocytes are generally matured in vitro before they are used as recipient cells for nuclear transfer. In preferred embodiments, this process generally involves collecting immature (prophase I) oocytes from mammalian ovaries, e.g., bovine ovaries obtained at a slaughterhouse, and maturing the oocytes in a maturation medium prior to until the oocyte attains the metaphase II stage, which in the case of bovine oocytes generally occurs about 18-24 hours post-aspiration. This period of time is known as the “maturation period.”

Melaphase II stage oocytes, which have been matured in vivo have also been successfully used in nuclear transfer techniques. Essentially, mature metaphase II oocytes are collected surgically from either non-superovulated or superovulated mammals (e.g. cows or heifers 35 to 48 hours) past the onset of estrus or past the injection of human chorionic gonadotropin (hCG) or similar hormone.

In general, successful mammalian embryo cloning practices use the metaphase II stage oocyte as the recipient oocyte because at this stage it is believed that the oocyte can be, or is, sufficiently “activated” to treat the introduced nucleus as it does a fertilizing sperm. In domestic animals, and especially cattle, the oocyte activation period generally ranges fiom about 16-52 hours, preferably about 28-42 hours post-aspiration.

For example, immature oocytes may be washed in HEPES buffered hamster embryo culture medium (HECM) as described in Seshagine et al. (1989) Biol. Reprod., 40, 544-606, and then placed into drops of maturation medium consisting of 50 microliters of tissue culture medium (TCM) 199 containing 10% fetal calf serum which contains appropriate gonadotropins such as luteinizing hormone (LH) and follicle stimulating hormone (FSH), ar.d estradiol under a layer of lightweight paraffin or silicon at 39° C.

After a fixed time maturation period, which ranges from about 10 to 40 hours, and preferably abcut 16-18 hours, the oocytes will be enucleated. Prior to enucleation the oocytes are preferably be removed and placed in HECM containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384. For example, metaphase II oocytes are either placed in HECM, optionally containing 7.5 μg/ml cytochalasin B, for immediate enucleation, or may be placed in a suitable medium, for example an embryo culture medium such as CR1aa, plus 10% estrus cow serum, and then enucleated later, preferably not more than 24 hours later, and more preferably 16-18 hours later.

Entuleation can also be accomplished microsurgically, e.g., using a micropipette to remove the polar body and the adjacent cytoplasm. The oocytes can then be screened to identiiy those of which have been successfully enucleated. This screening can be effected by staining the oocytes with 1 μg/ml 33342 Hoechst dye in HECM, and then viewing the oocytes under ultraviolet irradiation for less than 10 seconds. The oocytes that have been successfully enucleated can then be placed in a suitable culture medium, e.g., CR1aa plus 10% serum.

In somatic cell nuclear transfer, the recipient oocytes are preferably enucleated at a time ranging from about 10 hours to about 40 hours after the initiation of in vitro maturation, rnore preferably from about 16 hours to about 24 hours after initiation of in vitro maturation, and most preferably about 16-18 hours after initiation of in vitro maturation.

A single mammalian cell of the same species as the enucleated oocyte is then transferred into the perivitelline space of the enucleated oocyte used to produce the nuclear transfer unit (NT unit). The mammalian cell and the enucleated oocyte is used to produce NT units according to methods known in the art. For example, the cells can be fused by electrofusion.

Electrofusion is accomplished by providing a pulse of electricity that is sufficient to cause a transient and brief breakdown of the plasma membrane. If two adjacent membranes are induced to breakdown and upon reformation the lipid bilayers intermingle, small channels open between the two cells. Due to the thermodynamic instability of such a small opening, it enlarges until the two cells become one. Reference is made to U.S. Pat. No. 4,997,384 by Prather et al., for a further discussion of this process. A variety of electrofusion media can be used including e.g., sucrose, mannitol, sorbitol and phosphate buffered solution. Fusion can also be accomplished using Sendai virus as a fusogenic agent (Graham (1969) Inot. Symp. Monogr., 9:19).

Also, in some cases (e.g. with small donor nuclei) it may be preferable to inject the nucleus directly into the oocyte rather than using electroporation fusion. Such techniques are disclosed, for example in Collas and Barnes (1994) Mol. Reprod. Dev., 38:264-267.

After fusion, the resultant fused NT units are then placed in a suitable medium until activation, e.g., CR1aa medium. Typically activation will be effected shortly thereafter, typically less than 24 hours later, and preferably about 4-9 hours later.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions to which embryos are normally exposed.

Alternatively, activation may be achieved by application of known activation agents. For example, penetration of oocytes by sperm during fertilization has been shown to activate prefusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720.

Additionally, activation can be effected by simultaneously or sequentially increasing levels of divalent cations in the oocyte, and/or reducing phosphorylation of cellular proteins in the oocyte. This is generally effected by introducing divalent cations (e.g., magnesium, strontium, barium or calcium, preferably in the form of an ionophore) into the oocyte cytoplasm. Other methods of increasing divalent cation levels include the use of electric shock, treatment with ethanol and treatment with caged chelators.

Phosphorylation can be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins can be inhibited by introduction of a phosphatase into the oocyte, e.g., phosphatase 2A and phosphata,se 2B.

In one embodiment, NT activation is effected by briefly exposing the fused NT unit to a TL-HEPES medium containing 5 μM ionomycin and 1 mg/ml BSA, followed by washing in TL-HEPES containing 30 mg/ml BSA within about 24 hours after fusion, and preferably about 4 to 9 hours after fusion.

The activated NT units can then be cultured in a suitable in vitro culture medium until the generation of CICM cells and cell colonies. Culture media suitable for culturing and maturation of embryos are well known in the art. Examples of known media, which may be used for bovine embryo culture and maintenance, include, but are not limited to, Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% fetal calf serum, 0.2 mM Na pyruvate and 50 μg/ml gentamicin sulphate. Any of the above may also involve co-culture with a vaiety of cell types such as granulosa cells, oviduct cells, BRL cells and uterine cells and STO cells.

Another maintenance medium is described in U.S. Pat. No. 5,096,822. This embryo medium, named CR1, contains the nutritional substances necessary to support an embryo. CR1 contains hemicalcium L-lactate in amounts ranging from 1.0 mM to 10 mM, preferably 1.0 mM to 5.0 mM. Hemicalcium L-lactate is L-lactate with a hemicalcium salt incorporated thereon. Hemicalcium L-lactate is significant in that a single component satisfies two major requirements in the culture medium: (i) the calcium requirement necessary for compaction and cytoskeleton arrangement; and (ii) the lactate requirement necessary for metabolism and electron transport. Hemicalcium L-lactate also serves as valuable mineral and energy source for the medium necessary for viability of the embryos.

Advantageously, CR1 medium does not contain serum, such as fetal calf serum, and does not require the use of a co-culture of animal cells or other biological media, i.e., media comprising animal cells such as oviductal cells. Biological media can sometimes be disadvantageous in that they may contain microorganisms or trace factors which may be harmful to the embryos and which are difficult to detect, characterize and eliminate.

Examples of the main components in CR1 medium include hemicalcium L-lactate, sodium chloride, potassium chloride, sodium bicarbonate and a minor amount of fatty-acid free bovine serum albumin (Sigma A-6003). Additionally, a defined quantity of essential and non-essential amino acids may be added to the medium. CR1 with amino acids is known by the abbreviation “CR1aa.”

In one embodiment, the activated NT embryos unit are placed in CR1aa medium containing; 1.9 mM DMAP for about 4 hours followed by a wash in HECM and then cultured in CR1aa containing BSA.

For example, the activated NT units may be transferred to CR1aa culture medium containing 2.0 mM DMAP (Sigma) and cultured under ambient conditions, e.g., about 38.5° C., 5% CO.sub.2 for a suitable time, e.g., about 4 to 5 hours.

Afterward, the cultured NT unit or units are preferably washed and then placed in a suitable media, e.g., CR1aa medium containing 10% FCS and 6 mg/ml contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells. In one embodiment, the feeder cells comprise mouse embryonic fibroblasts.

The NT units are cultured on the feeder layer until the NT units reach a size suitable for transferring to a recipient female, or for obtaining cells which may be used to produce CICM cells or cell colonies. Preferably, these NT units will be cultured until at least about 2 to 400 cells, more preferably about 4 to 128 cells, and most preferably at least about 50 cells. The culturing will be effected under suitable conditions, i.e., about 38.5° C. and 5% CO₂, with the culture medium changed in order to optimize growth typically about every 2-5 days, preferably about every 3 days.

The methods for embryo transfer and recipient animal management for somatic cell nuclear transfer are standard procedures used in the embryo transfer industry. Synchronous transfers are important for success of the somatic cell nuclear transfer, i.e., the stage of the NT embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in Vitro (1981) L. Mastroianni, Jr. and J. D. Biggers, ed., Plenum Press, New York, N.Y., page 323).

Somatic cell nuclear transfer can also be used to clone genetically engineered or transgenic mammals (e.g. Ttpa knockouts). As explained above, the present invention is advantageous in that transgenic procedures can be simplified by working with a differentiated cell source that can be clonally propagated. In particular, the differentiated cells used for donor nuclei have a desired gene inserted, removed or modified. Those genetically altered, differentiated cells are then used for nuclear transplantation with enucleated oocytes.

For production of CICM cells and cell lines, after NT units of the desired size are obtained, the cells are mechanically removed from the zone and are then used. This is preferably effected by taking the clump of cells which comprise the NT unit, which typically will contain at least about 50 cells, washing such cells, and plating the cells onto a feeder layer, e.g., irradiated fibroblast cells. Typically, the cells used to obtain the stem cells or cell colonies will be cbtained from the inner most portion of the cultured NT unit which is preferably at least 50 cells in size. However, NT units of smaller or greater cell numbers as well as cells from other portions of the NT unit may also be used to obtain ES cells and cell colonies. The cells are maintained in the feeder layer in a suitable growth medium, e.g., alpha MEM supplemented with 10% FCS and 0.1 mM .beta.-mercaptoethanol (Sigma) and L-glutamine. The growth medium is changed as often as necessary to optimize growth, e.g., about every 2-3 days.

F) Other Non-Human Animals Which May Be Used to Practice the Invention

Having shown that disruption of the Ttpa gene reduces α-TTP production and that α-TTP deficient animals are viable, one of skill will recognize that there are a wide number of animals including natural and transgenic animals that have other desirable phenotypes and that can be used to practice the invention. Preferred animals are mammals including, but not limited to cattle, goats, sheep, canines, felines, largomorphs, rodents, murines, primates, especially non-human primates), pigs, and the like.

Zygotes or ES cells from the Ttpa knockouts of this invention such animals can be used as embryonic target cells for introduction of other heterologous genes or knockout constructs. Alternatively somatic cells can be used as targets for the introduction of various heterologous expression cassettes or knockout constructs.

In other embodiments, the knockout animals of this invention can be can be cross-bred with other animals exhibiting various natural or induced pathologies. In various embodiments, the knockout animals of this invention are crossed with animals having one or more knockouts other than the Ttpa knockout.

In certain preferred embodiments, a transgenic non-human animal is bred that that includes a deficiency in Ttpa expression (e.g. a heterozygous or homozygous Ttpa knockout) and a deficiency in a second recombinantly disrupted gene. In particularly preferred embodiments, the second knocked out gene is a gene whose phenotype is associated with a pathology involving oxidative stress (e.g. cancer, atherosclerosis, neurological disease, etc.).

Preferred variants include, but are not limited to animals produced by crossing an animal with a disruption in the Ttpa gene to an animal that has a natural, or bred, or recombinantly introduced, susceptibility to atherosclerosis (e.g., an animal that shows reduced or elevated apo E expression). Animals having abnormal apo E expression can be produced by breeding, or can comprise a recombinantly disrupted apo E gene.

By making the appropriate crosses, each gene can be maintained in the resulting animal in either the homozygous or heterozygous state. The phenotype of each class of animal containing the disrupted Ttpa and apo E genes in homozygous or heterozygous states can then be analyzed.

By use of available stem cells or by somatic nuclear transfer methods as described above, other Ttpa-knockouts and/or Ttpa/apo E knockouts or other TTPA/second gene knockouts can be produce in any of a wide variety of animals. Such animals include, but are not limited to hamsters, rats, rabbits, canines, felines, equines, bovines, and non-human primates.

One of skill will recognize that targeting of a transgene to a Ttpa allele in other species is facilitated by knowledge of the sequence of Ttpa gene in the subject species in order to incorporate the appropriate targeting sequences. The structure and function of the Ttpa genes other species are well known or can easily be ascertained using well known techniques by those of skill in the art.

For example, sequences from the mouse, hamster or human may be used as probes to identify the corresponding gene in other species using techniques well known to those of skill in the art. Thus, for example, a genomic or cDNA library for the subject species may be produced following published procedures (see, for example, Young et al. (1983) Proc. Natl. Acad. Sci., USA, 170: 827-842; Frischauf et al. (1983) J. Mol. Biol. 170: 827-842; Sambrook et al. (1989) Molecular Cloning: A Laboratory Manual (2nd Ed., Vols. 1-3, Cold Spring Harbor Laboratory). The library may screened (i.e., in a Southern Blot) under conditions of reduced stringency with appropriate probes to segments of the Ttpa□ genes. Once segments of the Ttpa gene in the subject species are identified, sequencing of the entire gene may be accomplished using routine methods well known to those of skill in the art (see, for example, Sambrook supra).

Once the target sequences in the Ttpa gene of the subject species are identified, creation of the disrupting transgene is routine to one of skill as described above. One may simply insert a disrupting marker as described in detail in Example 1, or alternatively one may introduce various insertions, deletions, or mutations as described above in section. Transformation of the subject organism may be accomplished using one of the methods described above.

G) Kits

In still another embodiment, this invention provides kits for the production of animals, typically mammals, comprising a knockout (disruption) of one or both alleles of an a-tocopherol transfer protein gene (Ttpa) as described herein. Preferred kits include a nucleic acid construct comprising a gene (Ttpa) disruption, e.g. as described herein, flanked by (Ttpa) sequences. Kits, optionally, comprise devices and/or reagents (e.g. cells or cell lines, buffers, cell culture media, ES cells, ect.) to facilitate the production of knockout animals as described herein.

The kits, optionally, include instructional materials providing protocols for creating and/or maintaining the knockout animals of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following example is offered to illustrate, but not to limit the claimed invention.

### Example 1

### Increased Atherosclerosis in Hyperlipidemic Mice Deficient in α-Tocopherol Transfer Protein and Vitamin E

Materials and Methods

Generatiorn of α-TTP Knockout Mice.

A 14-kb 129/Sv genomic λ clone containing the Ttpa gene was isolated and subcloned into pBSSKII. A sequence replacement vector was constructed by PCR amplification and subcloning of the short (˜1.1 kb) and long (˜9.5 kb) arms of homologous α-TTP sequence into a modified version of pKSl oxPNT (Hanks et al. (1995) Science 269: 679-682). A lacZ expression cassette was also cloned into the 5′ untranslated region of Ttpa gene. The vector was used to generate targeted embryonic stem cells and mice (Meiner et al. (1996) Proc. Natl. Acad. Sci., USA, 93: 14041-14046). Heterozygous mice (Ttpa^{+/-}) were intercrossed to generate Ttpa^{-/-} mice. Wild-type (16-kb) and disrupted (7-kb) HindIII fragments were identified by hybridizing a 32P-labeled 450-bp probe (located 5′ of the short arm of homology) synthesized by PCR amplification with sense (5′-AGC CAG AGG CAG ACA CAT TTA GG-3′, SEQ ID NO: 1) and antisense (5′-GCT TTG AAT TCT ATA CTG AGG AAG G-3′, SEQ ID NO: 2) primers. Subsequent genotyping in mice was performed by PCR with primeirs A (5′-TGA GTG TGC GTG GGG CGG CGT CC-3′, SEQ ID NO: 3), B (5′-CTG TTT CCC AAC CAA TGG CCC C-3′, SEQ ID NO: 4), and C (5′-CAT TCA GGC TGC GCA ACT GTT GGG-3′, SEQ ID NO: 5) at 95° C. for 10 min, followed by 30-cycles of 95° C. for 30 s, 60° C. for 30 s, and 72° C. for 1 min. A and B amplify a ˜138-bp fragment from the wild-type allele, and A and C amplify a ˜266-bp fragment from the knockout allele. Alice initially studied were of a mixed (50% C57BL/6 and 50% 129/SvJae) genetic background. Immunoblots were performed with a polyclonal antiserum as described (Terasawa et al. (1999) J. Lipid Res. 40: 1967-1977).

Atherosclerosis Study Mice.

Ttpa^{-/-} mice were crossed with apo E^{-/-} mice (˜100% C57BL/6) to generate Ttpa^{+/+} apo E^{-/-}, Ttpa^{+/+} apo E^{-/-}, and Ttpa^{-/-} apo E^{-/-} mice (˜75% C57BL/6 and ˜25% 129Sv/Jae background). Females were used in this study. We selected n=20 per genotype set, based on power calculations, which assumed standard deviations approximately equal to the mean (found in many atherosclerosis studies with apo E knockout mice) and a power of 80% in detecting a 75% difference between the means at P=0.05 confidence levels. Mice were housed in a pathogen-free barrier facility (12 h /12 h light/dark cycle) and fed chow (Picolab Mouse Chow 20, Purina, St. Louis, Mo.) containing ˜99 IU of vitamin E/kg.

Blood and Tissue Biochemical Analysis.

At 30 weeks of age, blood was collected by cardiac puncture, the mice were perfused with phosphate-buffered saline, and tissues were harvested and frozen in liquid nitrogen. Cholesterol levels were measured by colorimetric assay (Spectrum, Abbott Laboratories). HDL cholesterol was quantified after the precipitation of the apo-B-containing lipoproiteins with polyethylene glycol-8000 (Purcell-Huynh et al. (1995) J. Clin. Invest. 95: 2246-2257).

Vitamin E was measured in plasma after extraction without saponification, a modified method of Lang et al. (1986) Anal. Biochem. 157: 106-116. Tissue vitamin E was extracted by a modified alcoholic KOH saponification procedure described by Podda et al. (Podda et al. (1996) J. Lipid Res. 37: 893-901). The HPLC system consisted of a Shimadzu (Kyoto, Japan) pump (LC-10ADVP), controller (SCL-10AVP), and an auto-injector (SIL-10ADVP), and a Waters Spherisorb ODS2 C-18 column (4.6 mm i.d., 100 mm, 3-μm particle size) and Spherisorb ODS precolumn (5 μm, 1 cm×4.6 mm). In addition, a LC-4C amperometric electrochemical detector (Bioanalytical Systems, Lafayette, Ind.) with a glassy carbon working electrode and a silver chloride reference electrode was used with an isocratic system. The electrochemical detector was in the oxidizing mode, potential 500 mV, full recorder scale at 500 nA. Shimadzu Scientific 4.2 Class VP software was used to integrate peak areas. Ascorbate and urate were measured by paired-ion reversed-phase HPLC coupled with electrochemical detection (Kutnink et al. (1987) Anal. Chem. 166: 424-430).

Atherosclerotic Lesion Analysis.

Female mice were killed at 30 weeks of age after 27 weeks of chow feeding. Blood was collected by cardiac puncture. Tissues were fixed by perfusion with 3% paraformaldehyde in phosphate buffer (pH 7.3), and aortas were removed, opened longitudinally from the heart to the iliac bifurcation, and pinned out flat (Palinski et al. (1995) Arterioscler. Thromb. Vasc. Biol. 15: 1569-1576). Aortic images were captured with a Polaroid digital camera (DMC1) mounted on a Leica Mz6 dissection microscope and analyzed with Adebe Photoshop 5.0.1 software and Image Processing Tool Kit (IPHSV-TK, Reindeer Games, Gainesville, Fla.) plug-ins. An image of each aorta was captured and divided into three regions (arch, thorax, and abdomen) from which both surface and lesion areas were quantifled. Percent lesion area results were calculated from lesion area and total surface area.

Aortic root morphology was examined in three Ttpa^{+/+} apo E^{-/-} and three Ttpa^{-/-} apo E^{-/-} mice, which had total aortic lesion areas representative of the means of each genotype. Aortic roots were fixed by perfusion with 3% paraformaldehyde in phosphate buffer (pH 7.3), embedded in OCT, frozen, sectioned, and stained with Movat's pentachrome.

Aortic α-Tocopherol and F2-Isoprostane Measurements.

Mice were killed at age 30 weeks and perfused with phosphate-buffered saline. Whole aortas were dissected and divided into two portions (proximal 2/3 and distal 1/3) for measurements of total F2-isoprostane and α-tocopherol levels. Aortas were immediately frozen in liquid nitrogen until analysis. Total F2-isoprostanes were measured as described (Awad et al. (1994) J. Nutr. 124: 810-816), and α-tocopherol was measured as described above.

Results

We created a genetic model of vitamin E deficiency by disrupting the mouse α-TTP gene (Ttpa) (FIGS. 1A and 1B). Immunoblotting of liver homogenates showed no α-TTP in Ttpa^{-/-} mice and decreased amounts in Ttpa^{+/-} mice (FIG. 1C). In chow-fed mice, α-tocopherol levels in plasma and most tissues were reduced by ˜50% in Ttpa^{+/-} mice (not shown) and more than 90% in Ttpa^{-/-} mice (FIG. 1D). In Ttpa^{-/-} liver, adipose tissue, adrenal gland, and aorta, α-tocopherol levels were 15-35% of those of wild-type mice. The reason for the higher α-tocopherol levels in these tissues is uncertain but may reflect the delivery and accumulation of dietary α-tocopherol from chylomicrons and their remnants.

Ttpa^{-/-} mice were generally healthy. Offspring from heterozygous intercrosses were born with the expected Mendelian distribution. Ttpa^{-/-} mice at 18 months of age had no obvious signs of neurological disease. In contrast, humans with α-TTP gene defects develop ataxia with vitamin E deficiency by the first decade of life (Sokol et al. (1988) J. Lab. Clin. Med. 111: 548-559; Ouahchi et al. (1995) Nat. Genet. 9: 141-145; Hentati et al. (1996) Ann. Neurol. 39: 295-300). This discrepancy may reflect species differences in the susceptibility of the nervous system to vitamin E deficiency (Follis (1958) pp 159-170 in Deficiency Disease: Functional and Structural Changes in Mammalia Which Result from Exogenous or Endogenous Lack of One or More Essential Nutrients, (Charles C. Thomas, Springfield, Ill.). Ttpa^{-/-} females were, however, infertile. This fertility defect presumably resulted from vitamin E deficiency. Vitamin E is required to prevent fetal resorption in rodents (Evans and Bishop (1922) Science 56: 650-651; Urner (1931) Anat. Rec. 50: 175-187), and vitamin E supplementation (1000 IU/kg of diet) completely reversed the fertility defect (not shown). Ttpa^{-/-} males had no obvious impairment in fertility.

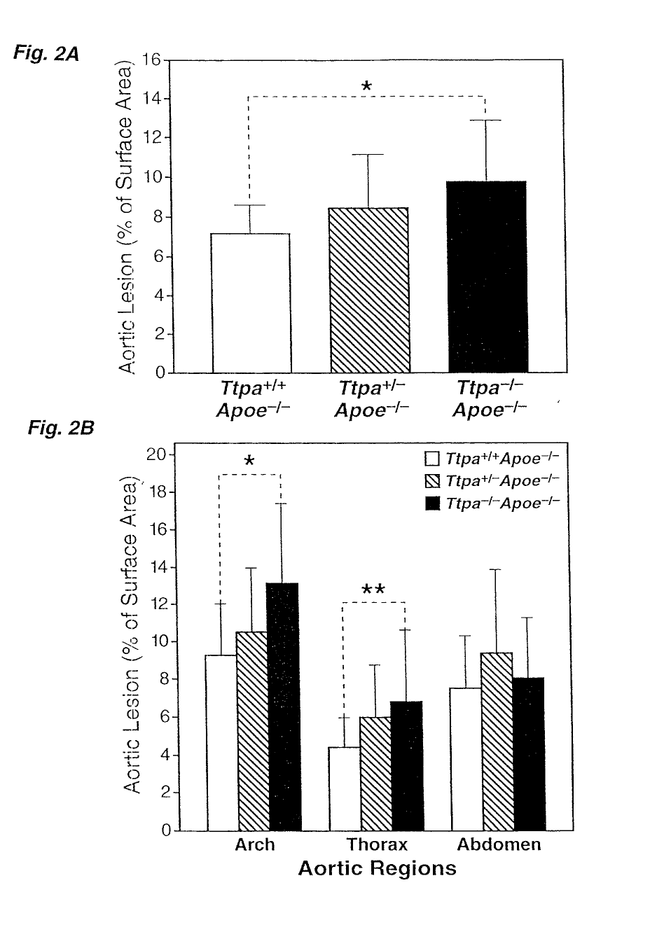

To investigate whether deficiency of α-TTP and α-tocopherol increased atherosclerosis, we crossed Ttpa^{-/-} mice with apo E^{-/-} mice (Piedrahita et al. (1992) Proc. Natl. Acad. Sci., USA, 89:4471-4475). In Ttpa^{-/-} apo E^{-/-} and Ttpa^{+/-} apo E^{-/-} mice, plasma α-tocopherol levels were 1.4% and 76%, respectively, of those in Ttpa^{+/+} apo E^{-/-} mice (FIG. 1D and Table 1). In most tissues of Ttpa^{-/-} apo E^{-/-} mice, including the proximal aorta, α-tocopherol levels were more than 85% lower than those in Ttpa^{+/+} apo E^{-/-} mice (FIG. 1D). apo E^{-/-} liver, adipose tissue, adrenal gland, and distal aorta. Plasma levels of α-tocopherol were low in all groups, probably reflecting the small amounts of α-tocopherol in the diet. Aortic atherosclerotic lesions were quantified in chow-fed Ttpa^{+/+} apo E^{-/-}, Ttpa^{+/-} apo E^{-/-}, and Ttpa^{-/-} apo E^{-/-} mice at 30 weeks of age. Total aortic lesion area was ˜36% greater in Ttpa⁻⁻ apo E^{-/-} mice than in Ttpa^{+/+} apo E^{-/-} controls (9.77±3.12 vs. 7.17±1.43% of surface area, P=0.005) (FIG. 2A). Aortic lesions in all groups were most severe in the aortic arch region (proximal ⅓ of aorta) (FIG. 2B). Compared with Ttpa^{+/+apo E}^{-/-} controls, Ttpa^{-/-} apo E^{-/-} had 42% larger lesions (P=0.002) and Ttpa^{+/-} apo E^{-/-} mice had 13% larger lesions (P=0.054) in the aortic arch. In the thorax region (middle ⅓ of aorta), Ttpa^{-/-} apo E^{-/-} mice had 53% more lesion area than Ttpa^{+/+} apo E^{-/-} mice (P=0.03). α-TTP deficiency did not affect lesion size in the abdominal (distal ⅓) aorta.

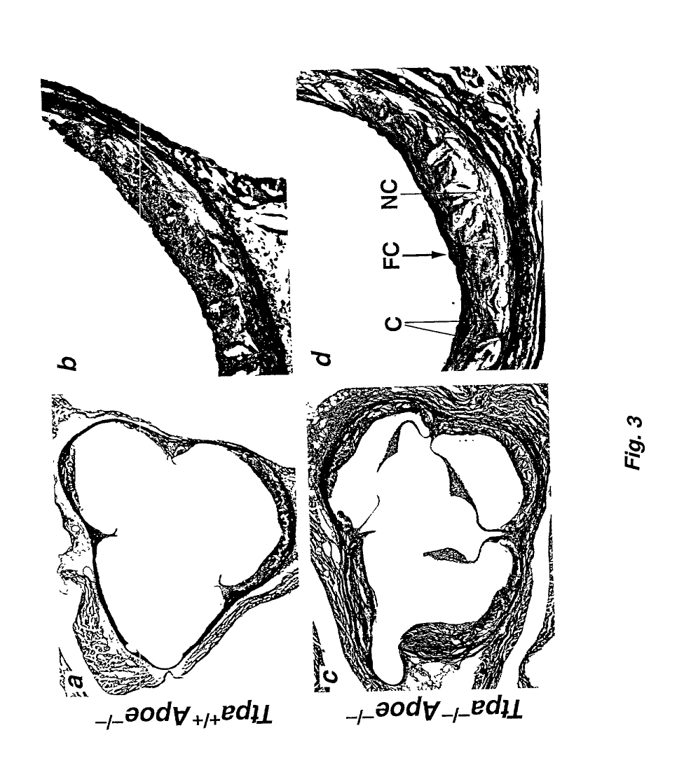

In a subset of mice, we examined the morphology of the aortic root lesions. Lesions of Ttpa^{-/-} apo E^{-/-} mice consistently appeared more complex than those of Ttpa^{+/+} apo E^{-/-} controls, with more area occupied by necrotic core and cholesterol crystals and some lesions having fibrous caps (FIG. 3). Macrophage immunostaining appeared similar in aortic root sections of the two groups of mice (not shown).

TABLE 1
Plasma levels of cholesterol and antioxidants.

| | Total | α- | | | |
|---|---|---|---|---|---|
| | Cholesterol | Tocopherol | γ-Tocopherol | Ascorbate | Urate |
| Genotypes | (mg/dl) | (μM) | (μM) | (μM) | (μM) |
| Ttpa^{+/+} apo E^{-/-} | 427.4 ± 143.7 | 11.9 ± 4.5 | 0.15 ± 0.06 | 71.3 ± 16.0 | 68.7 ± 25.7 |
| Ttpa^{+/-} apo E^{-/-} | 442.0 ± 91.3 | 9.0 ± 2.3 | 0.11 ± 0.04 | ND | ND |
| Ttpa^{-/-} apo E^{-/-} | 433.1 ± 113.9 | 0.17 ± 0.09 | 0.01 ± 0.00 | 81.3 ± 24.4 | 6.5 ± 10.1 |

Data are presented as mean±SD. Plasma cholesterol levels were measured at the time the mice were killed for atherosclerotic lesion quantitation (20 Ttpa^{+/+} apo E^{-/-}, 19 Ttpa^{+/-} apo E^{-/-}, and 21 Ttpa^{-/-}apo E^{-/-} female mice). Plasma α-tocopherol and α-tocopherol levels were measured in these study mice and others for a total of 30 Ttpa^{+/+} apo E^{-/-}, 19 Ttpa^{+/-} apo E^{-/-}, and 32 Ttpa^{-/-} apo E^{-/-} mice. Plasma α-tocopherol levels for Ttpa^{+/+} apo E and Ttpa^{-/-} apo E^{-/-} mice are also shown in FIG. 1D. Ascorbate and urate levels were measured from a subset of mice (eight Ttpa^{+/+} apo E^{-/-} and nine Ttpa^{-/-} apo E^{-/-} mice). * P<0.05 vs. Ttpa^{+/+} apo E^{-/-} or Ttpa^{+/-} apo E^{-/-}, ANOVA with Dunn's test; † P−0.004 vs. Ttpa^{+/+} apo E^{-/-}, ANOVA with Tukey test; ‡ P<0.001 vs. Ttpa^{+/+} apo E^{-/-} or Ttpa^{+/-} apo E^{/-}, ANOVA with Tukey test. ND, not determined.

To establish that the differences in atherosclerotic lesion development did not result from differences in plasma levels of cholesterol or antioxidants other than vitamin E, we measured total cholesterol, high density lipoprotein (HDL) cholesterol, ascorbate, and urate in the plasma. Total plasma cholesterol levels were similar in mice of all genotypes (Table 1), as were HDL cholesterol levels (22.5±5.8 vs. 22.8±7.5 mg/dl for Ttpa^{+/+} apo E^{-/-} and Ttpa^{-/-} apo E^{-/-} mice, respectively). In addition, cholesterol distribution in the lipoprotein fractions assessed by fast protein liquid chromatography was similar for Ttpa^{+/+} apo E^{-/-} and Ttpa^{-/-} apo E^{-/-} mice (not shown). Plasma ascorbate and urate levels were also similar in both groups (Table 1).

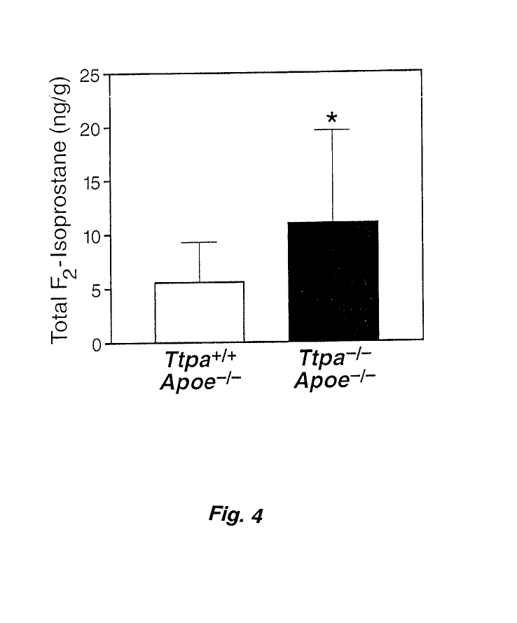

To analyze the relationship between lesion development and lipid peroxidation, we measured aortic levels of total F2-isoprostanes, a marker of lipid peroxidation Morrow and Roberts (1997) Prog. Lipid Res. 36: 1-21), in separate groups of Ttpa^{+/+} apo E^{-/-} and Ttpa^{-/-} apo E^{-/-} mice. Total F2-isoprostanes in the proximal aorta, where α-tocopherol levels were sevenfold reduced in Ttpa^{-/-} apo E^{-/-} mice (FIG. 1D), were nearly twofold higher in Ttpa^{-/-} apo E^{-/-} mice than Ttpa^{+/+apo E}^{-/-} controls (11.32±8.78 vs. 5.93±3.71 ng/g, n=10 for each genotype, P=0.03) (FIG. 4). Total F2-isoprostane levels were also nearly twofold higher in distal aortas of Ttpa^{-/-} apo E^{-/-} mice than Ttpa^{+/+} apo E^{-/-} controls (27.3±2.1 vs. 14.8±3.5 ng/g, n=6 for each genotype, P=0.002), despite the less pronounced difference between α-tocopherol levels.

Discussion

In human and animal studies, the ability of vitamin E supplementation to prevent atherosclerosis (Upston, et al. (1999) FASEB J. 13: 977-994; Yusuf et al. (2000) N. Engl. J. Med. 342: 154-160; Chan (1998) J. Nutr. 128: 1593-1596; Keaney et al.(1999) FASEB J. 13: 965--976; Praticò et al. (1998) Nat. Med. 4: 1189-1192; Shaish et al. (1999) Arterioscler. Thromb. Vasc. Biol. 19: 1470-1475) has varied, possibly because of differences in vitamin E supplementation regimens, other dietary factors, or the degree of preexisting atherosclerosis. Uniquely, our study examines the effect of vitamin E deficiency on atherogenesis as a single modifying factor present before lesion development. Our results indicate that α-TTP deficiency and associated vitamin E deficiency promote lesion formation in the proximal aorta in the setting of increased susceptibility to atherosclerosis, in this case, caused by apo E deficiency. Thus, vitamin E deficiency appears to modulate, rather than cause, atherosclerosis. Supporting this, we have not observed spontaneous atherosclerosis in normolipidemic α-TTP-deficient mice that have apo E (Y. Terasawa and R. Farese, unpublished observations). Similarly, early onset atherosclerosis has not been reported in humans with α-TTP gene defects (Cavalier et al. (1998) Am. J. Hum. Genet. 62: 301-310).

The increase in atherosclerotic lesion area in aortas was significant (increased by 35% to 40% in α-tocopherol-deficient mice) but not dramatic. Several factors may have accounted for this. First, although α-tocopherol levels were reduced in Ttpa^{-/-} apo E^{-/-} aortas, substantial amounts of α-tocopherol were present in this tissue, possibly due to the delivery of α-tocopherol from dietary lipoproteins, which circulate at high levels in apo E-deficient mice (Piedrahita et al. (1992) Proc. Natl. Acad. Sci., USA, 89:4471-4475). Lesion area might have been greater if α-tocopherol levels had been more severely reduced. Second, the lesion smalysis method we employed (whole aorta analysis) may have minimized differences. apo E-deficient mice tend to develop prominent lesions in the aortic root (Nakashima et al. (1994) Arterioscler. Thromb. 14: 133-140). Cross-sectional analysis of lesion area in aortic roots therefore might have resulted in greater lesion areas and amplified any differences due to α-tocopherol deficiency. Finally, compensatory changes in other antioxidant systems might have mitigated the effects of α-tocopherol deficiency on lesion development. For example, deficiency of paraoxonase, an HDL-associated enzyme with antioxidant properties, results in increased atherosclerosis in apo E-deficient mice and is associated with upregulation of hepatic expression of heme oxygenase-1, possibly to compensate for the increase in oxidative stress (Shih et al. (2000) J. Biol. Chem. 275: 17527-17535).

The major effects of α-TTP and α-tocopherol deficiency on lesion formation were observed in the proximal two-thirds of the aorta. In this region, a sevenfold reduction in α-tocopherol levels was associated with a 35% to 40% increase in lesion areas. These results are consistent with those of Praticò et al. (1998) Nat. Med. 4: 1189-1192, who showed that vitamin E supplementation of apo E^{-/-} mice fed a chow diet resulted in decreased levels of atherosclerosis. Why α-tocopherol deficiency had no effect on atherosclerosis in the distal aortas of our study mice is unknown. α-Tocopherol levels in the distal aortas of Ttpa^{-/-} apo E^{-/-} mice were only reduced by ˜60% compared with Ttpa ^{+/+} apo E^{-/-} controls, perhaps accounting for the lack of effect. Another possibility is that the effects of vitamin E on lesion development may vary with anatomical location. It is noteworthy that probucol, a potent lipid-soluble antioxidant, did not prevent progression of femoral artery lesions in a human clinical trial (Walldius et al.(1994) Am. J. Cardiol. 74: 875-883) nor of lesion development in abdominal aortas or iliac arteries of nonhuman primates (Sasahara et al. (1994) J. Clin. Invest. 94:155-164).

Decreased lipid peroxidation is a likely mechanism by which vitamin E prevents atherosclerotic lesion formation. We therefore examined the relationship between aortic α-tocopherol and F2-isoprostane levels. In the proximal aorta, reduced tissue α-tocopherol levels wvere associated with a twofold increase in F2-isoprostanes. These results are consistent with those of Praticò et al. (1998) Nat. Med. 4: 1189-1192, who found that apo E^{-/-} mice fed a chow diet had higher levels of a subset of F2-isoprostanes (iPF2_{2α}-VI) in their aortas than apo E^{-/-} mice fed a diet supplemented with vitamin E. In our study, the increase in F2-isoprostane levels in the proximal aorta was associated with increased lesion areas. However, we did not find increased lesion areas in the distal aortas of Ttpa^{-/-} apo E^{-/-} mice, despite a twofold increase in aortic F2-isoprostanes. This suggests either that factors other than lipid peroxidation contribute to lesion formation in this region or that the content of F2-isoprostanes in this tissue at the end of the study period may not accurately reflect the oxidant status during lesion formation. Although our data generally support the hypothesis that vitamin E reduces atherosclerosis through its antioxidant properties, the mechanism by which vitamin E affects atherosclerosis development was not directly addressed by our study, and other mechanisms (reviewed in Chan (1998) J. Nutr. 128: 1593-1596; Keaney et al.(1999) FASEB J. 13: 965-976; Traber and Packer (1995) Am. J. Clin. Nutr. 62: 1501S-1509S) might have contributed. We also cannot exclude the possibility that the increased atherosclerosis in Ttpa^{-/-} apo E^{-/-} mice resulted from an effect of α-TTP deficiency other than reduced α-tocopherol levels. We believe this is unlikely, however, because the only known function of α-TTP is in vitamin E metabolism.

The Ttpa^{-/-} mice provide a new and exciting genetic model of vitamin E deficiency. Plasma and tissue α-tocopherol levels are reduced in a step-wise and consistent manner in Ttpa^{+/-} and Ttpa^{-/-} mice. In the present study, we used this model to address the role of vitamin E and oxidative stress on atherosclerosis, but Ttpa^{-/-} mice will likely prove valuable for studying other diseases in which lipid peroxidation or antioxidants may play a role.

It is understood that the examples and embodiments described herein are for illustrative purposes only and that various modifications or changes in light thereof will be suggested to persons skilled in the art and are to be included within the spirit and purview of this application arid scope of the appended claims. All publications, patents, and patent applications cited herein are hereby incorporated by reference in their entirety for all purposes.

What is claimed is:

1. A knockout mammal, said mammal comprising a disruption in an endogenous α-tocopherol transfer protein gene (Ttpa), wherein said disruption results in said knockout mammal exhibiting a decreased level of α-tocopherol transfer protein α-TTP) as compared to a wild-type animal.

2. The mammal of claim 1, wherein the mammal is selected from the group consisting of an equine, a bovine, a rodent, a porcine, a lagomorph, a feline, a canine, a murine, a caprine, an ovine, and a non-human primate.

3. The mammal of claim 1, wherein the disruption is selected from the group consisting of an insertion, a deletion, a frameshift mutation, a substitution, and a stop codon.

4. The mammal of claim 3, wherein the disruption comprises an insertion of an expression cassette into the endogenous Ttpa gene.

5. The mammal of claim 4, wherein said expression cassette comprises a selectable marker.

6. The mammal of claim 4, wherein the expression cassette comprises a neomycin phosphotransferase gene operably linked to at least one regulatory element.

7. The mammal of claim 4, wherein the expression cassette is inserted into exon 1 of the endogenous Ttpa gene.

8. The mammal of claim 2, wherein said disruption is in a somatic cell.

9. The mammal of claim 2, wherein said disruption is in a germ cell.

10. The mammal of claim 2, wherein the mammal is homozygous for the disrupted Ttpa gene.

11. The mammal of claim 2, wherein the mammal is heterozygous for the disrupted Ttpa gene.

12. The mammal of claim 2, wherein said mammal further comprises a second recombinantly disrupted gene.

13. The mammal of claim 12, wherein said second gene comprises a disruption that prevents the expression of a functional polypeptide from said disrupted second gene.

14. The mammal of claim 13, wherein the mammal is homozygous for said disrupted second gene.

15. The mammal of claim 13, wherein the mammal is heterozygous for said disrupted second gene.

16. The mammal of claim 12, wherein the second gene is selected from the group consisting of an apo E gene, and an APP gene.

17. A mammalian model of atherosclerosis, said model comprising a rodent comprising:
a disruption in an endogenous α-tocopherol transfer protein gene (Ttpa), wherein said disruption results in said knockout rodent exhibiting decreased levels of α-tocopherol transfer protein α-TTP) as compared to a wild-type animal; and
wherein said rodent exhibits reduced expression of apo E as compared to a healthy wildtype rodent of the same species.

18. The mammalian model of claim 17, wherein said rodent is the F1 progeny of a cross between a rodent comprising a disruption in an endogenous α-tocopherol transfer protein gene and a mammal showing reduced expression of apo E as compared to a healthy wildtype rodent of the same species.

19. The mammalian model of claim 17, wherein said rodent is heterozygous for a disruption in an endogenous α-tocopherol transfer protein gene.

20. The mammalian model of claim 17, wherein said rodent is homozygous for a disruption in an endogenous α-tocopherol transfer protein gene.

21. The mammalian model of claim 17, wherein said rodent comprises a disruption in an endogenous apo E gene, wherein said disruption results in said knockout rodent exhibiting decreased levels of apo E as compared to a wild-type animal.

22. The mammalian model of claim 21, wherein said rodent is homozygous for said disruption in an endogenous apo E gene.

23. The mammalian model of claim 21, wherein said rodent is homozygous for said disruption in an endogenous apo E gene.

24. The mammalian model of claim 21, wherein said rodent is homozygous for said disruption in an endogenous α-tocopherol transfer protein gene and homozygous for said disruption in an endogenous apo E gene.

25. The rodent of claim 17, wherein the rodent is a mouse.

26. The rodent of claim 17, wherein the disruption is selected from the group consisting of an insertion, a deletion, a frameshift mutation, a substitution, and a stop codon.

27. A knockout rodent comprising a disruption in an endogenous α-tocopherol transfer protein gene (Ttpa) wherein said disruption results in said knockout rodent exhibiting cecreased levels of α-tocopherol transfer protein α-TTP) as compared to a wild-type animal.

28. The rodent of claim 27, wherein the rodent is a mouse.

29. The rodent of claim 27, wherein the disruption is selected from the group consisting of an insertion, a deletion, a frameshift mutation, and a stop codon.

30. The rodent of claim 27, wherein the disruption comprises an insertion of an expression cassette into the endogenous Ttpa gene.

31. The rodent of claim 30, wherein the expression cassette comprises a selectable marker.

32. The rodent of claim 30, wherein the expression cassette comprises a neomycin phosphctransferase gene operably linked to at least one regulatory element.

33. The rodent of claim 30, wherein the expression cassette is inserted into exon 1 of the endogenous Ttpa gene.

34. The rodent of claim 27, wherein said disruption is in a somatic cell.

35. The rodent of claim 27, wherein said disruption is in a germ cell.

36. The rodent of claim 27, wherein the rodent is homozygous for the disrupted Ttpa gene.

37. The rodent of claim 27, wherein the rodent is heterozygous for the disrupted Ttpa gene.

38. The rodent of claim 27, wherein said rodent further comprises a second recombinantly disrupted gene.

39. The rodent of claim 38, wherein said second gene comprises a disruption and wherein said disruption prevents the expression of a functional product from said disrupted second gene.

40. The rodent of claim 39, wherein the rodent is homozygous for said disrupted second gene.

41. The rodent of claim 39, wherein the rodent is heterozygous for said disrupted second gene.

42. The second gene of claim 39, wherein the second gene is selected from the group consisting of an apo E gene, and an APP gene.

43. A nucleic acid for disrupting an α-tocopherol transfer protein gene, said nucleic acid comprising:
α-tocopherol transfer protein gene sequences that undergo homologous recombination with an endogenous α-tocopherol transfer protein gene; and
a nucleic acid sequence that, when introduced into an α-tocopherol transfer protein gene inhibits expression of said α-tocopherol transfer protein gene.

44. The nucleic acid of claim 43, wherein said nucleic acid when introduced into an α-tocopherol transfer protein gene creates a disruption selected from the group consisting of an insertion, a deletion, a frameshift mutation, and a stop codon.

45. The nucleic acid of claim 44 wherein the disruption comprises an insertion of an expression cassette into the endogenous Ttpa gene.

46. The nucleic acid of claim 45, wherein said expression cassette comprises a selectable marker.

47. The nucleic acid of claim 46, wherein the expression cassette comprises a neomycin phosphotransferase gene operably linked to at least one regulatory element.

48. The nucleic acid of claim 43, wherein said nucleic acid comprises Ttpa nucleic acid sequences flanking a nucleic acid encoding a Ttpa disruption.

49. The nucleic acid of claim 48, wherein said nucleic acid is present in a vector.

50. A nucleic acid comprising a nucleic acid encoding a disrupted α-tocopherol transfer protein gene (Ttpa) wherein the disruption prevents the expression of a functional α-tocopherol transfer protein α-TTP) from said nucleic acid.

51. The nucleic acid of claim 50, wherein said nucleic acid comprises a disruption selected from the group consisting of an insertion, a deletion, a frameshift mutation, and a stop codon.

52. The nucleic acid of claim 50, wherein said nucleic acid is a deoxyribonucleic acid (DNA).

53. The nucleic acid of claim 50, wherein said nucleic acid is in a mammalian cell.

54. A mammalian cell comprising a disruption in an endogenous α-tocopherol transfer protein gene (Ttpa) wherein said disruption results in said cell exhibiting decreased levels of α-tocopherol transfer protein α-TTP) as compared to a wild-type animal.

55. The cell of claim 54, wherein said cell of a mammal is selected from the group consisting of an equine, a bovine, a rodent, a porcine, a lagomorph, a feline, a canine, a murine, a caprine, an ovine, and a non-human primate.

56. The cell of claim 54, wherein the cell is a rodent cell.


SEQUENCE LISTING (5 sequences)

SEQ ID NO: 1 - DNA, 23 bp, Artificial Sequence
  PCR primer
agccagaggcagacacatttagg

SEQ ID NO: 2 - DNA, 25 bp, Artificial Sequence
  PCR primer
gctttgaattctatactgaggaagg

SEQ ID NO: 3 - DNA, 23 bp, Artificial Sequence
  PCR primer
tgagtgtgcgtggggcggcgtcc

SEQ ID NO: 4 - DNA, 22 bp, Artificial Sequence
  PCR primer
ctgtttcccaaccaatggcccc

SEQ ID NO: 5 - DNA, 24 bp, Artificial Sequence
  PCR primer
cattcaggctgcgcaactgttggg